(19) United States
(12) Patent Application Publication
(10) Pub. No.: US 2014/0081182 A1
(43) Pub. Date: Mar. 20, 2014

(54) METHOD AND APPARATUS FOR DETERMINING AT LEAST ONE PREDETERMINED MOVEMENT OF AT LEAST ONE PART OF A BODY OF A LIVING BEING

(71) Applicant: Robert Bosch GmbH, Stuttgart (DE)

(72) Inventors: Hans-Peter Klose, Stuttgart (DE); Anna-Karina Grebe, Marbach (DE); Kerstin Steidle, Fellbach (DE)

(73) Assignee: Robert Bosch GmbH, Stuttgart (DE)

(21) Appl. No.: 14/030,836

(22) Filed: Sep. 18, 2013

(30) Foreign Application Priority Data
Sep. 19, 2012 (DE) 10 2012 216 747.1

(51) Int. Cl. A61B 5/11

(52) U.S. Cl. CPC A61B 5/1118
USPC 600/595

(57) ABSTRACT

The disclosure relates to a method for determining at least one predetermined movement of at least one part of a body of a living being. The method includes reading in movement data representing a movement at least of the at least one part of the body of the living being and reading in comparison data from a memory, the comparison data representing a predetermined movement of the at least one part of the body. The method also includes comparing the movement data with the comparison data. Furthermore, the method includes recognizing the predetermined movement of the at least one part of the body if the movement data are in a predetermined relationship with respect to the comparison data in order to determine the movement of the at least one part of a body of the living being.

This application claims priority under 35 U.S.C. §119 to patent application no. DE 10 2012 216 747.1, filed on Sep. 19, 2012 in Germany, the disclosure of which is incorporated herein by reference in its entirety.

### BACKGROUND

The present disclosure relates to a method for determining at least one predetermined movement of at least one part of a body of a living being, to a corresponding apparatus and to a corresponding computer program product.

Numerous motion and activity sensors (for example Aipermon, Fitbit, etc.) which use different approaches to record movements of persons/patients are nowadays on the market. Those devices which record the movement of the person using single-axis, two-axis or three-axis acceleration sensors (for example produced using MEMS technology) are widespread, in particular in the upper price segment. In medical practice, particular tests are carried out under supervision in the prior art in order to assess the patient status, for example:
a six-minute walk test (6MWT) in which furthermore the distance walked can also be evaluated (COPD);
a timed-up-and-go test (TUG) in which the time between standing up and a 3 m walk is evaluated in order to assess the risk of falling; or
assessment of an arm movement while a patient is drinking coffee in order to detect Parkinson's disease.

US 2011 009 23 37 A1 discloses a portable system for monitoring strength training.

### SUMMARY

Against this background, the present disclosure presents a method for determining at least one predetermined movement of at least one part of a body of a living being, as well as an apparatus which uses this method and finally a corresponding computer program product. Advantageous refinements emerge from the following description.

The present disclosure provides a method for determining at least one predetermined movement of at least one part of a body of a living being, the method having the following steps:
reading in movement data representing a movement at least of the part of a body of the living being and reading in comparison data from a memory, the comparison data representing a predetermined movement of the at least one part of the body;
comparing the movement data with the comparison data;
recognizing the predetermined movement of the at least one part of the body if the movement data are in a predetermined relationship with respect to the comparison data in order to determine the movement of the at least one part of a body of the living being.

A predetermined movement can be understood as meaning a movement which is carried out according to a predefined movement pattern or sequence. A body of a living being may be understood as meaning the body of an animal or a human. A part of a body may be understood as meaning, for example, an extremity such as an arm or a leg, a hand, a foot, the head or else the torso of the living being. Movement data may be understood as meaning data which have been currently recorded, for example, and represent a movement of the part of the body of the living being, for example in digitized form. For example, the movement data may represent different positions of the part of the body of the living being at different times during a movement sequence of a movement of the part of the body of the living being. Alternatively or additionally, the movement data may represent different accelerations of the part of the body of the living being at different times during a movement sequence of the movement of the part of the body of the living being. Comparison data may be understood as meaning, for example, digital data which are read in from a memory and represent a predefined movement of the at least one part of the body and were recorded at a time before the recording time of the movement data. For example, the comparison data may represent different positions of the part of the body of the living being at different times when the part of the body of the living being carries out the predefined movement. Alternatively or additionally, the comparison data may also represent different accelerations of the part of the body of the living being at different times when the part of the body of the living being carries out the predefined movement. Alternatively or additionally, the comparison data may also represent threshold values and limit values which can be used to classify a movement of a part of the body of a living being. In this case, the comparison data are stored in a memory and can be used as a reference for how the part of the body of the living being should or must behave during the predetermined movement.

Comparison of the movement data with the comparison data may be understood as meaning a process in which individual items of information from the movement data, which are assigned to predetermined times, or threshold values valid for this time, are related to items of information from the comparison data which are assigned to the corresponding times of the comparison data. In this respect, a plurality of individual comparison operations, in each of which an item of information from the movement data is compared with an item of information from the comparison data, can be carried out in the comparison step, the items of information compared in each case corresponding to the same times in the movement data or the comparison data. A predetermined relationship may be understood as meaning, for example, that the comparison data have a larger or smaller value than the movement data or that a value, which is formed by linking the comparison data to the movement data (for example by means of addition, subtraction, multiplication or division), is greater or less than a threshold value. Recognition of the predetermined movement of the at least one part of the body can be understood as meaning identification of this predetermined movement when a parameter representing a match between the movement data and the comparison data, for example, is not greater than a predetermined threshold value. Such a parameter can be formed, for example, by virtue of the fact that a difference is formed in the comparison step when comparing each item of information from the movement data with a corresponding item of information from the comparison data and the sum of the individual differences is then formed in order to form the parameter.

The present disclosure is based on the knowledge that a predetermined movement of a part of the body can be recognized when the currently recorded movement data are compared with comparison data which have previously been recorded or determined in another manner and represent the predetermined movement of the part of the body. In this case, it is possible to recognize in a very efficient manner which movement has been carried out by precisely the living being using a part of its body by comparing the movement data which have been read in with the previously stored comparison data. For example, when the arm is moved, a characteristic pattern of an acceleration or a position of the arm can be recognized over the course of time, with the result that, if there is a great match between the movement data and this pattern from the comparison data, the movement of the body part which is actually carried out can now be identified.

The present disclosure therefore affords the advantage of being able to automatically recognize an actual movement of a body part (that is to say a part of the body) of the living being. There is therefore no need for a supervisor, such as medical professionals, to be present when identifying the movement in order to register and detect a movement which is actually carried out by the body part of the living being. The present disclosure therefore makes it possible to recognize movements of a body part of the living being even in the absence of the supervisor and/or over a very long period which cannot be monitored by a supervisor. In this case, it is particularly advantageous if, in one embodiment of the present disclosure, an item of information relating to the recognized movement of the part of the body of the living being is also stored (for example in the memory). In this case, a time and/or an environmental situation of the living being, for example temperature, humidity, a noise level or a similar variable, may also be linked, for example, to this information relating to the recognized movement of the part of the body and stored. This information can then be conveniently read from the memory at a subsequent time.

The present disclosure also provides an apparatus which is designed to carry out, control or implement the steps of the method according to the disclosure in corresponding devices or units. In particular, the present disclosure therefore provides an apparatus for determining at least one predetermined movement of at least one part of a body of a living being, the apparatus having the following features:
an interface for reading in movement data representing a movement at least of the part of a body of the living being and for reading in comparison data from a memory, the comparison data representing a predetermined movement of the at least one part of the body;
a comparison unit which is designed to compare the movement data with the comparison data; and
a recognition unit which is designed to recognize the predetermined movement of the at least one part of the body if the movement data are in a predetermined relationship with respect to the comparison data in order to determine the movement of the at least one part of a body of the living being.

This embodiment variant of the disclosure in the form of an apparatus can also be used to quickly and efficiently achieve the object on which the disclosure is based.

In the present case, an apparatus may be understood as meaning an electrical device which processes sensor signals and outputs control and/or data signals on the basis thereof. The apparatus may have an interface which may be designed using hardware and/or software. In the case of a design using hardware, the interfaces may be, for example, part of a so-called system ASIC which comprises a wide variety of functions of the apparatus. However, it is also possible for the interfaces to be separate, integrated circuits or to consist at least partially of discrete components. In the case of a design using software, the interfaces may be software modules which are present, for example, on a microcontroller in addition to other software modules.

A computer program product with program code which may be stored on a machine-readable carrier, such as a semiconductor memory, a hard disk memory or an optical memory, and is used to carry out the method according to one of the embodiments described above when the program product is executed on a computer or an apparatus is also advantageous.

It is particularly advantageous if, according to one embodiment of the present disclosure, movement data which at least partially represent an acceleration of the at least one part of the body are read in in the reading-in step. Such an embodiment of the present disclosure affords the advantage that accelerations can be measured in a very simple, reliable and cost-effective manner using currently available sensors and precise detection and recognition of the movement of the body part is therefore possible.

According to another embodiment of the present disclosure, movement data which represent a temporal course of movement of the at least one part of the body during a predetermined period can be read in in the reading-in step. A predetermined period may be understood as meaning, for example, a period which comprises several seconds to several minutes or hours. Such an embodiment of the present disclosure affords the advantage that very precise recognition of the movement of the body part which is actually carried out is possible by evaluating the movement data which contain an item of information relating to a movement of the body part over a temporally extended course of movement. The more information relating to the movement of the body part there is, that is to say the longer the period over which movement data are available, the better and more precise the estimation or recognition of the movement of the body part which is actually carried out.

According to another embodiment of the present disclosure, the steps of the method can also be carried out repeatedly during a predetermined period of time, in particular during a period of time of several hours and/or days. Such an embodiment of the present disclosure affords the advantage of recognizing a plurality of identical movements of the body part which are carried out at different times in this period of time. In this case, it is also conceivable for the comparison data to be calibrated, that is to say, for example in the case of movement data which have occurred repeatedly in this period of time and have made it possible to recognize a particular movement of the body part, for these movement data to be stored as comparison data in the memory and for the memory to thus be updated with respect to the comparison data and/or to be personalized for the relevant living being in each case.

An embodiment of the present disclosure in which at least one characteristic variable, in particular a distance walked by a person and/or a deviation of the movement data from the comparison data, is also determined from the movement data during the recognition step and/or the comparison step is also particularly favorable. A characteristic variable may be understood as meaning, for example, a variable which is obtained by processing the movement data alone and/or by relating the comparison data to the movement data. Such a characteristic variable may be, for example, a variable which represents the deviation of the movement data from the comparison data. Such an embodiment of the present disclosure affords the advantage of using movement data which are already available to obtain further information. In this case, there is no need for a supervisor, such as medical professionals, to be available, which also reduces costs by saving work. At the same time, such technical evaluation of the movement data may result in greater accuracy of the determined characteristic variable than would be the case as a result of estimation or calculation by a supervisor.

It is also particularly advantageous if, according to one embodiment of the present disclosure, an outputting step is also provided, in which a request for a predetermined movement to be carried out by a person is output and/or in which a movement of the at least one part of the body, as determined in the recognition step and/or the comparison step, and/or the determined characteristic variable is/are output. Such an embodiment of the present disclosure affords the advantage that it is not only possible to passively determine or recognize a movement carried out by the body part but also to actively request a predetermined movement of a body part of the living being to be carried out. Such a request may involve, for example, requesting the person to lift a leg or to move the arm according to a predetermined pattern, for example to draw a circle or to guide a cup to the mouth. Alternatively or additionally, the possibility of outputting a determined movement or a determined characteristic variable makes it possible to transmit an item of information relating to the kind and/or type of movement of the body part (even over a relatively long period of time) and/or corresponding deviations to a supervisor (for example medical staff) in a short time. In this case, it is also possible to output, for example, an item of information relating to a time of the recognized or determined movement of the body part and/or the determined characteristic variable if said item of information has been stored together with the recognized or determined movement of the body part and/or the determined characteristic variable. As a result, the supervisor can very easily gain an overview of the movements carried out by the living being over the course of time.

An embodiment of the present disclosure in which a response period between output of the request for the predetermined movement to be carried out by the person and the reading-in of the movement data is also determined in the reading-in step is also advantageous, in which case, in particular, the predetermined movement is recognized in the recognition step when the response period is inside a predefined response time tolerance range. A response time tolerance range may be understood as meaning, for example, a time range from half a second to 5 seconds. In such a response time tolerance range, it can usually be assumed that a healthy patient will carry out the requested predetermined movement. Such an embodiment of the present disclosure affords the advantage that a further parameter relating to the condition of the living being is available by evaluating the response period and may be of interest, in particular, in medical examinations. As a result of the interaction (presented here) between the living beings and an apparatus which implements an embodiment of the method proposed here, it is therefore possible to collect data which can be collected, on the one hand, over a very long period of time and, on the other hand, can be collected without the direct presence of medical staff and are therefore considerably closer to reality than data acquired in a short interval of time in a laboratory or a doctor's surgery.

An embodiment of the present disclosure in which a step of providing the movement data is also provided before the reading-in step is also advantageous, in which the movement of the at least one part of the body with respect to at least one further part of the body and/or with respect to an object in an environment of the body is determined and provided. Such an embodiment of the present disclosure affords the advantage of collecting the movement data in a technically very simple manner and providing said data for evaluation. For this purpose, it is possible to use, in particular, acceleration sensors and/or distance sensors (for example for the three-dimensional detection of a position of the sensor) in order to directly detect the acceleration of the body part or the position of the sensor or capital when the sensor has been directly fastened to the body part.

### BRIEF DESCRIPTION OF THE DRAWINGS

The disclosure is explained by way of example in more detail below using the accompanying drawings, in which:

FIG. 1 shows a basic diagram of the use of the present disclosure, a block diagram of an apparatus according to one exemplary embodiment of the present disclosure being illustrated;

FIG. 2 shows a graph illustrating movement data which can be used to determine the predetermined movement;

FIG. 3 shows a block diagram of a movement detection system in which an exemplary embodiment of the present disclosure can be used;

FIG. 4 shows a diagram of an exemplary scenario for acquiring movement data when a predefined movement is carried out by a person;

FIG. 5 shows a diagram of an exemplary scenario for explaining the determination of a characteristic variable and a conclusion on a patient status which can be drawn from the characteristic variable;

FIG. 6 shows a diagram of an exemplary scenario for acquiring movement data after outputting a request for a predetermined movement; and

FIG. 7 shows a flowchart of a method according to one exemplary embodiment of the present disclosure.

### DETAILED DESCRIPTION

In the following description of preferred exemplary embodiments of the present disclosure, identical or similar reference symbols are used for the elements which are illustrated in the different figures and act in a similar manner, in which case a repeated description of these elements is dispensed with.

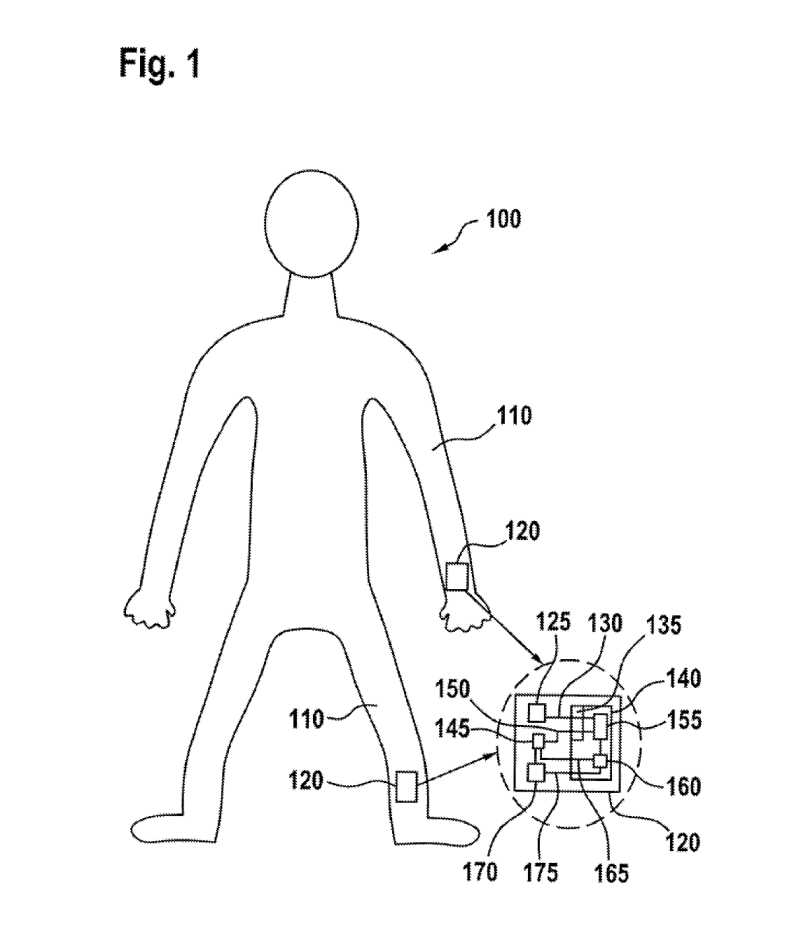

FIG. 1 shows a basic diagram of the use of the present disclosure, a block diagram of an apparatus according to one exemplary embodiment of the present disclosure being illustrated. In this case, FIG. 1 illustrates a living being 100, here a person, having an apparatus 120 for determining at least one predetermined movement on at least one body part 110. Instead of the persons 100 illustrated in FIG. 1, another living being, for example an animal, can also be provided with the corresponding apparatus 120. The apparatus 120 can be fastened to the body part 110, for example a leg and/or an arm, using a fastening strap, for example. As is illustrated in more detail in the detail excerpt at the lower right of FIG. 1, a sensor 125, for example, which determines an acceleration of the apparatus 120 or a distance between the apparatus 120 and a reference apparatus (for example another apparatus 120 which is fastened to the torso of the living being or to another body part of the living being) can be arranged in the apparatus 120. Alternatively or additionally, one or more further variables which represent or characterize the movement of the body part 110 may naturally also be determined by the sensor 125. The sensor 125 therefore provides movement data 130 which are provided, for example, in the form of a data sequence, this data sequence being able to represent a temporal profile of a variable recorded by the sensor 125. The movement data 130 are transmitted to a data processing unit 140 using an interface 135. Comparison data 150 are transmitted from a memory 145 to the data processing unit 140 using the interface 135. These comparison data 150 may be data which are characteristic of a predetermined movement of a body part 110, for example may contain a temporal profile of acceleration values, which are typically experienced by a leg, as the body part 110, during walking, and/or threshold values which are characteristic of this movement. Alternatively or additionally, the comparison data 150 may also represent a temporal profile of a distance from the apparatus 120 or the sensor 125 experienced by the leg, as the body part 110, when walking over a distance. A similar situation naturally likewise applies to a movement of another body part 110, for example an arm. In this case, the comparison data 150 naturally have a different temporal profile of the variable which can be recorded by the sensor 125 if the body part were to carry out a movement corresponding to the comparison data. In this respect, the comparison data 150 stored in the memory 145 can be used to characterize a predetermined movement of the body part 110 of the living being 100 and are then used to identify a movement actually carried out by the body part 110 using the movement data 130 acquired by the sensor 125.

The movement data 130 and the comparison data 150 are now compared with one another in a comparison unit 155 in the data processing unit 140. If the movement data 130 differ from the comparison data 150 by no more than a suggested tolerance range (for example by no more than 10 percent), it is also possible to recognize, in a recognition unit 160, that the movement which was carried out by the body part 110 and resulted in the movement data 130 provided by the sensor 125 actually corresponds to the predetermined movement to which the comparison data 150 are assigned. In this case, the recognition unit 160, for example, can store an item of movement information 165 in the memory 145, said information stating that the body part 110 has carried out a movement assigned to the comparison data 150. In addition, an item of time information relating to the time at which the carrying-out of the movement assigned to the comparison data 150 was recognized by the recognition unit 160 may also be integrated in the movement information 165, for example.

Furthermore, an input/output interface 170 may also be provided in the apparatus 120, which interface is designed to output the movement information 165 (possibly together with the associated time information) stored in the memory 145 to a user of the apparatus 120. Alternatively or additionally, the input/output interface 170 may also be designed to store further comparison data 150 assigned to other movements of the relevant body part 110 in the memory 145. In this case, the movement data 130 can be compared with a respective set of a plurality of different comparison data items 150 (each assigned to different movements) in the comparison unit 155, for example, in order to recognize whether the body part 110 has carried out one of the movements assigned to the different comparison data items 150. Furthermore, it is also conceivable for the input/output interface 170 to be designed to output a request to the living being 100, for example a person, to have a (predetermined) movement of the body part 110 carried out by the living being 100. It is likewise possible for the input/output interface 170 to output a timing start signal 175 to the recognition unit 160 at the same time as a request for a predetermined movement of the capital of another living being 100 in order to start timing. The timing (for example in the recognition unit 160) is ended, for example, when the recognition unit 160 recognizes that the requested predetermined movement of the body part 110 has been carried out. In this case, that time at which the movement recognized by the recognition unit 160 has begun may be determined as the starting time for the requested movement to be carried out. Evaluating this response time of the living being 100, as recorded by the timing, likewise makes it possible to determine an additional item of information for a user of the apparatus 120, for example. This response time may likewise be stored together with the movement information 165 in the memory 145, for example.

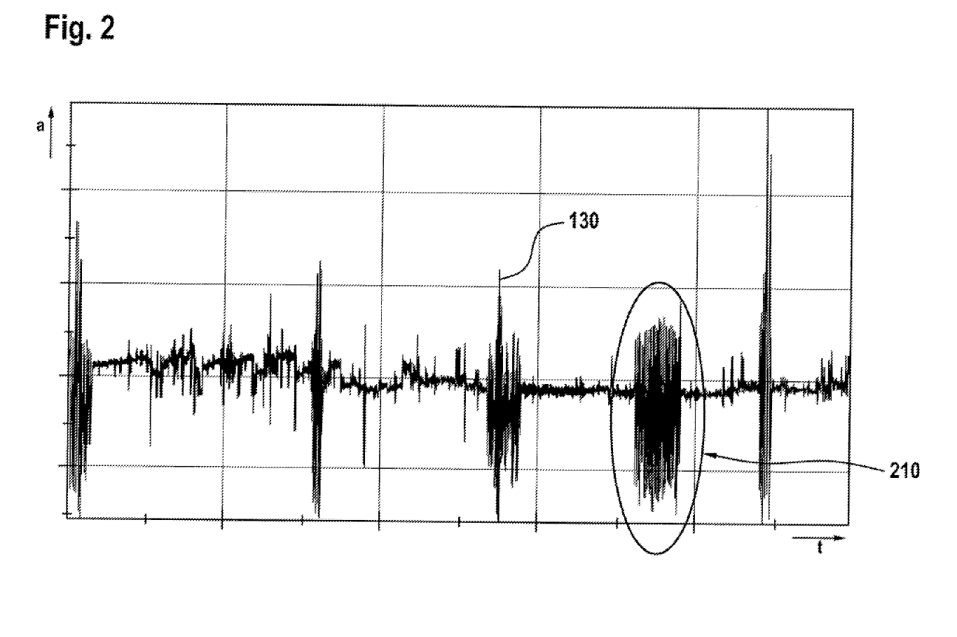

FIG. 2 shows a graph illustrating movement data 130 which can be used to determine the predetermined movement. In this case, the movement data 130 depict, for example, an item of information relating to what acceleration acts on the body part 110 or the distance between the body part 110 and a reference position. In this case, the movement data are plotted as a temporal profile, that is to say the information or physical variable which is current at the respective times is recorded as a movement data item 130 at different times. The movement data 130 are now compared with the comparison data 150. In this case, a section of the movement data 130 may be singled out and may be compared with the comparison data 150. This singling-out may be effected, for example, using different criteria, for example a section of the movement data 130 which is longer than a predetermined period and in which the movement data 130 exceed a predetermined threshold value (for example in terms of magnitude). This may be an indication, for example, of the fact that the body part 110 is actively moved during this longer period. FIG. 2 illustrates such a section 210. This period of time which is the longest continuous period in the entire measurement signal can therefore be a period in which a patient 100 walks a particular distance. Comparing this section 210 of the movement data 130 with the comparison data 150 makes it possible to now verify that, for example, the leg, as the body part 110 of the patient 100, is moving back and forth according to a walking movement, as is stored as a typical pattern in the comparison data 150. If the step length of the patient 100, for example, is also known, the distance can also be determined, as a characteristic variable, from the movement data 130 in the section 210 if walking of the patient 100 is recognized and can be stored, for example, as, or in addition to, corresponding information 165 in the memory 145. This then also makes it possible to determine, for example, a parameter in the form of the distance, which parameter can be compared with a walking test which is manually carried out but can be determined in a considerably simpler and more cost-effective manner.

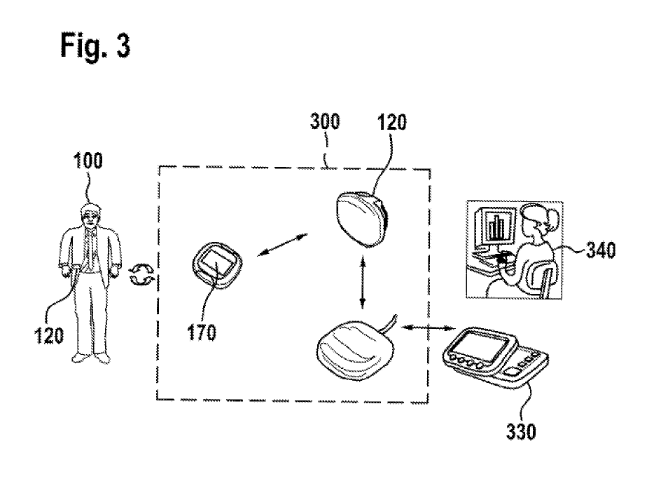

FIG. 3 shows a block diagram of a movement detection system 300 in which an exemplary embodiment of the present disclosure can be used. The movement detection system 300, which may be partially fastened to a patient 100 for example, may and grasp the apparatus 120 which can be interpreted as a sensor device and is fastened to the body as a data recording device. This apparatus 120 may likewise communicate with the input/output unit 170 in order to display information to the patient or the person 100 or medical staff or to output requests to the patient 100 for movements of the body part 110. Furthermore, the movement detection system 300 may have, for example, a further apparatus 120 which can be used as a reference unit which is fastened to the patient 100 at another position and makes it possible, for example, to detect a distance between the apparatus 120 and the reference unit 120, for example via a wireless data connection. Alternatively, the reference unit 120 may also be understood as meaning a readout device in a doctor's surgery, by means of which device recorded data in the memory 145 of the apparatus 120 can be read out (for example in a wired manner) and can be transmitted to a monitoring station 330. This monitoring station 330 can be used, for example in a telemedical system, to transmit the data which have been read out to a doctor 340 or another medical professional who can draw conclusions on the condition of the patient 100 from these data. The monitoring station 130 can furthermore also be used as a charging station in order to charge an energy store of the apparatus 120 or of the reference unit 120.

The statement of the tests carried out under supervision and in the presence of the medical staff could be improved, in the manner proposed here, by means of further tests in the domestic environment. For this purpose, the motion sensor can therefore be used with sequence recognition. Said sensor recognizes the abovementioned tests/sequences on a daily basis and evaluates them. As an example of a use of the approach presented here, the following movement data acquisition scenarios are conceivable:
Are there continuous walking episodes? If so: how long do they last? What was the longest walking episode? What associated distance was covered in this time? What distance was covered in a six-minute walk test? This corresponds to a conventional domestic six-minute walk test but can now be carried out without the presence of medical professionals.
What do standing up and starting look like in the domestic environment (inter alia a controlled TUG test)?
Do continuous movements (for example walking) above a particular threshold (for example time, energy consumption etc.) occur with the patient?
What does the arm movement look like in the domestic environment in a patient with Parkinson's disease?
What walking pattern can be observed in patients with Parkinson's disease, etc.?

The evaluation of the automatically recognized movement sequences results in objectified evaluation of the movements of patients over a longer period of time. Changes between measurements with an interval of several weeks/months can thus be better recognized than when the evaluation is carried out only by eye.

The data recorded by the motion sensor are analyzed by means of an algorithm which is able

1) to recognize particular predefined movement sequences (for example walking faster than 1.0 m/s without interruption) and
2) to calculate likewise predefined characteristic variables (for example average speed, energy consumption etc. or duration of a movement, regularity of performance etc.) for this identified section. If necessary, the characteristic variables calculated from the recognized sequences can also be directly compared with reference values which were predefined by the doctor as “training objectives”, for example.

The calculation should advantageously be carried out on the motion sensor worn by the patient. The device 120 is intended to have an interface 135 or 170 which is used:

1) to define sequences and evaluation characteristic variables (and to write these to the memory of the apparatus);
2) to make it possible to read out the evaluation results. The treating specialist can then directly read the results from the device (for example as statistics, a daily profile or a weekly profile) after the device has been worn for four weeks, for example.

The advantage of this new sequence analysis over the prior art is that, in addition to the tests recorded selectively at the doctor's surgery, actual daily movements can be analyzed and evaluated. This has the advantage that the patient's movements are not imposed and the evaluation, assessment and possibly therapy can therefore be effected closer to reality. This is the longest continuous walking period in the entire measurement signal.

If the distance walked is calculated over the period (for example 6 minutes), a parameter which can be compared with the 6-MWT carried out manually is obtained.

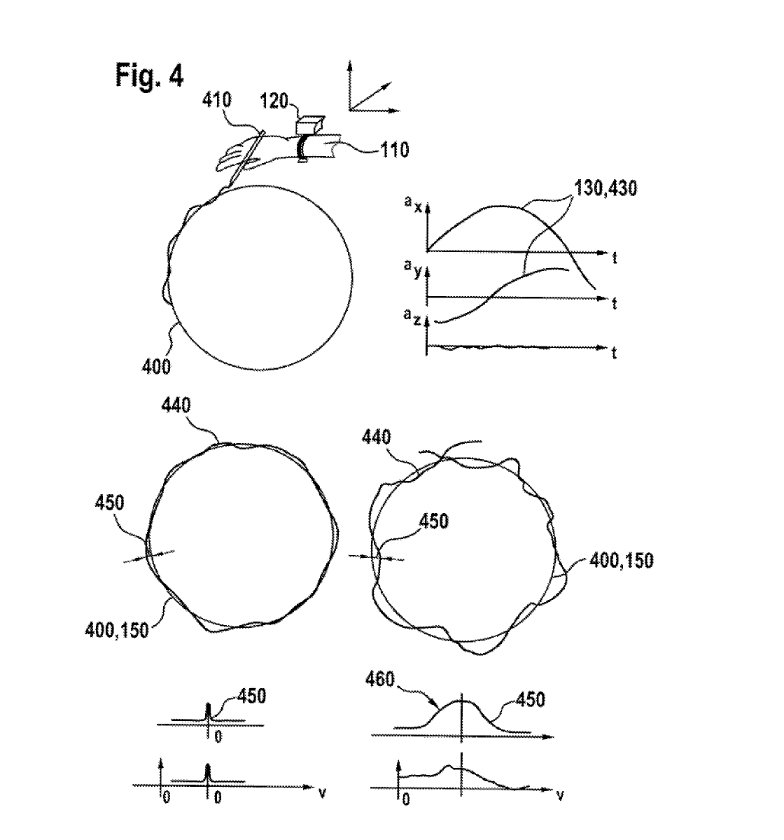

FIG. 4 shows a diagram of an exemplary scenario for acquiring movement data when a predefined movement is carried out by a person. In this case, the illustration from FIG. 4 shows the procedure for acquiring the movement data. For example, the patient 100 may be caused to move his relevant body part 110, such as his arm or his hand here, in such a manner that he traces a predefined circle 400 with a pen 410. The sensor in the apparatus 120 can then be used, for example, to record an acceleration value aₓ in the direction of the x axis illustrated in FIG. 4, an acceleration value a_{y} in the direction of the y axis and an acceleration value a_{z} in the direction of the z axis, as reproduced in the illustration at the top right of FIG. 4. A conclusion on the condition of the patient can now be drawn from these movement data which are now present in the form of accelerations of the body part 110 in different directions. If the patient is healthy, for example, the evaluation of the movement data 130 in the form of the acceleration values 430 could allow a conclusion that the circle 440 traced by the patient differs only very slightly from the predefined circle 400 to be traced. This slight difference may be recognized, for example, by a short (averaged) distance 450 between the circle 440 drawn by the patient and the predefined circle 400. Alternatively or additionally, the slight difference may also be recognized by the fact that the uniformity is determined using a drawing speed v which is usually very constant and uniform with a healthy patient when following the circle 400, as reproduced in the illustration at the bottom left.

In contrast, a patient with Parkinson's disease, for example, will be able to trace the predefined circle 400 only with a considerably greater difference 450. In a statistical evaluation of all segments of the circle 400 or 440, this variable of the difference 450 has an effect such that a considerably broader distribution curve 460 than in the case of healthy patients results. The drawing speed will also be considerably more inconsistent than in healthy patients. This is illustrated in the illustration at the bottom right of FIG. 4.

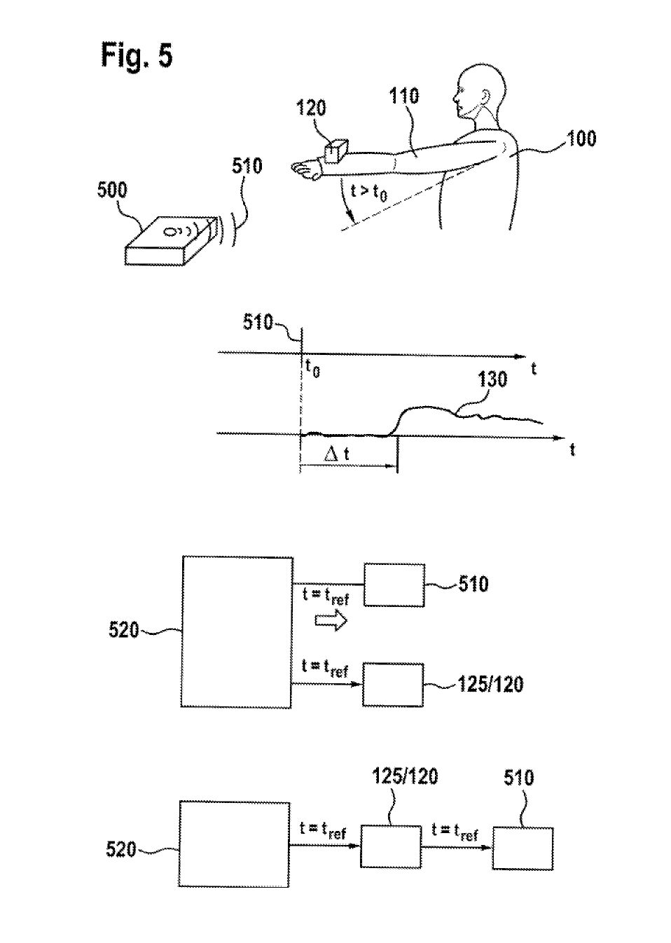

FIG. 5 shows an illustration for explaining the determination of a characteristic variable and a conclusion on a patient status which can be drawn from the characteristic variable. In this case, a starting generator 500, for example, can be used to transmit a start signal 510 to the patient 100, for example in an acoustic or optical manner. This starting generator 500 may be, for example, part of the apparatus 120 or of the input and output unit 170. In this case, the start signal 510 may request the patient 100, for example, to lift a particular body part 110, the arm here. The sensor in the apparatus 120 then makes it possible to record, for example, the accelerations which occur when lifting the arm 110, a time difference between the output of the start signal 510 and the start of the process of lifting the arm 110 also being determined at the same time. If the start signal 510 is now output at the time t₀, an actual movement of the arm can usually be recognized in the movement data 130 after a certain delay Δt. This delay can now likewise give an indication of the condition of the patient 100 and/or can be stored together with the movement data 130 and/or a recognized movement of the body part 110.

A pre-requisite for such an exemplary embodiment of the present disclosure should be sufficiently good synchronization of the starting generator with the unit for detecting the movement, for example the sensor 125 or the apparatus 120. This synchronization may be effected, for example, in such a manner that the reference time t_{ref} is generated or provided in an input and output unit 170 and is transmitted to the starting generator 500 and to the acceleration sensor 125 or the apparatus 120. Alternatively, it is also conceivable for the reference time to first of all be transmitted from the input and output unit 170 to the acceleration measuring unit 125 or 120 and to be transmitted from the acceleration measuring unit 120/120 to the starting generator 510.

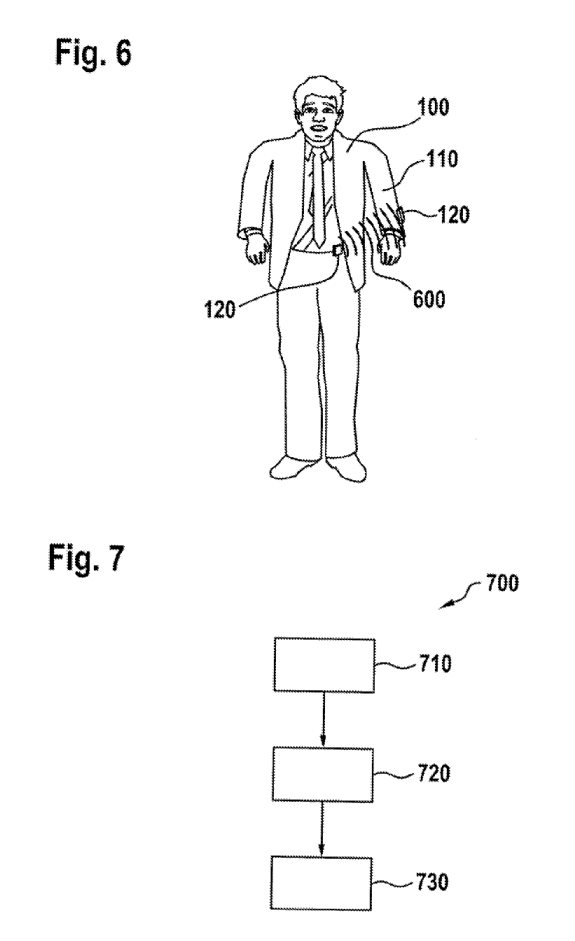

FIG. 6 shows an illustration of an exemplary scenario for acquiring movement data after outputting a request for a predetermined movement. According to the exemplary embodiment from FIG. 6, the apparatus 120 may be fastened to the arm or directly to the stomach of the patient 100 and may synchronize the reference time, for example, using a wireless connection 600. In this case, the start signal 510 may also be output, for example, by the apparatus 120 fastened to the stomach of the patient 100, whereas the movement of the body part 110 is detected and recognized by the apparatus 120 fastened to the arm. In this case, the movement of the arm may also be detected in the apparatus 120, for example, by determining a distance between the apparatus 120 on the arm and the apparatus 120 on the stomach of the patient, which is a possible procedure for detecting the movement of the arm 110, as an alternative to detecting the accelerations of the arm 110.

FIG. 7 shows a flowchart of a method 700 for determining at least one predetermined movement of at least one part of a body of a living being according to one exemplary embodiment of the present disclosure. The method 700 comprises a step of reading in 710 movement data representing a movement at least of the part of a body of the living being and reading in comparison data from a memory, the comparison data representing a predetermined movement of the at least one part of the body. The method 700 also comprises a step of comparing 720 the movement data with the comparison data and a step of recognizing 730 the predetermined movement of the at least one part of the body if the movement data are in a predetermined relationship with respect to the comparison data in order to determine the movement of the at least one part of a body of the living being.

The exemplary embodiments described and shown in the figures are selected only by way of example. Different exemplary embodiments may be combined with one another entirely or with respect to individual features. An exemplary embodiment can also be supplemented with features of a further exemplary embodiment.

Method steps according to the disclosure may also be carried out repeatedly and in a sequence other than the described sequence.

If an exemplary embodiment comprises an “and/or” conjunction between a first feature and a second feature, this should be read in such a manner that the exemplary embodiment has both the first feature and the second feature according to one embodiment and has either only the first feature or only the second feature according to another embodiment.

What is claimed is:

1. A method for determining at least one predetermined movement of at least one part of a body of a living being, comprising:
reading in (i) movement data representing a movement at least of the at least one part of the body of the living being, and (ii) comparison data from a memory, the comparison data representing a predetermined movement of the at least one part of the body;
comparing the movement data with the comparison data; and
recognizing the predetermined movement of the at least one part of the body if the movement data are in a predetermined relationship with respect to the comparison data in order to determine the movement of the at least one part of the body of the living being.

2. The method according to claim 1, wherein the movement data at least partially represent an acceleration of the at least one part of the body.

3. The method according to claim 1, wherein the movement data represent a temporal movement sequence of the at least one part of the body during a predetermined period.

4. The method according to claim 1, wherein:
the method is carried out repeatedly during a predetermined period of time, and
the predetermined period of time is several hours and/or several days.

5. The method according to claim 1, wherein:
at least one characteristic variable is also determined from the movement data during at least one of (i) the recognizing the predetermined movement, and (ii) the comparing the movement data, and
the at least one characteristic variable is at least one of a distance walked by a person and a deviation of the movement data from the comparison data.

6. The method according to claim 1, further comprising:
outputting at least one of (i) a request for a predetermined movement to be carried out by a person, (ii) a movement of the at least one part of the body, as determined in the recognizing the predetermined movement and/or the comparing the movement data, and (iii) the determined characteristic variable.

7. The method according to claim 6, wherein a response period between the output of the request for the predetermined movement to be carried out by the person and the reading-in of the movement data is also determined in the reading-in, in which case, the predetermined movement is recognized in the recognizing the predetermined movement when the response period is inside a predefined response time tolerance range.

8. The method according to claim 1, further comprising:
also providing the movement data before the reading-in, in which a movement of the at least one part of the body with respect to at least one of (i) at least one further part of the body, and (ii) an object in an environment of the body is determined and provided.

9. An apparatus for determining at least one predetermined movement of at least one part of a body of a living being, comprising:
an interface configured (i) to read-in movement data representing a movement at least of the at least one part of the body of the living being, and (ii) to read-in comparison data from a memory, the comparison data representing a predetermined movement of the at least one part of the body;
a comparison unit configured to compare the movement data with the comparison data; and
a recognition unit configured to recognize the predetermined movement of the at least one part of the body if the movement data are in a predetermined relationship with respect to the comparison data in order to determine the movement of the at least one part of the body of the living being.

10. A computer program product, comprising:
a program code configured to carry out a method for determining at least one predetermined movement of at least one part of a body of a living being when the program product is executed on an apparatus, the method including
using an interface to read in (i) movement data representing a movement at least of the at least one part of the body of the living being, and (ii) comparison data from a memory, the comparison data representing a predetermined movement of the at least one part of the body,
comparing the movement data with the comparison data using a comparison unit, and
using a recognizing unit to recognize the predetermined movement of the at least one part of the body if the movement data are in a predetermined relationship with respect to the comparison data in order to determine the movement of the at least one part of the body of the living being.
